(19) United States
(12) Patent Application Publication
(10) Pub. No.: US 2007/0037295 A1
(43) Pub. Date: Feb. 15, 2007

(54) Method for preparing a sample

(76) Inventors: Mattias Vangbo, Fremont, CA (US); Marten Stjernstrom, Danderyd (SE); Monica Veszelei, Uppsala (SE); Anders Tangen, Uppsala (SE)

Correspondence Address: GE HEALTHCARE BIO-SCIENCES CORP.;PATENT DEPARTMENT, 800 CENTENNIAL AVENUE, PISCATAWAY, NJ, 08855, US

(21) Appl. No.: 10/558,919

(22) PCT Filed: Mar. 24, 2005
(86) PCT No.: PCT/EP05/03160

§ 371 Date: Nov. 30, 2005

(30) Foreign Application Priority Data
Mar. 27, 2004 (GB) 0406953.0

(51) Int. Cl. G01N 33/558; G01N 33/50

(52) U.S. Cl. 436/514; 436/86

(57) ABSTRACT

The present invention relates to a method for reducing the solvent content of a sample in a microchannel or other microfluidic structure comprising the steps of 1) freezing said sample and 2) exposing the frozen sample to a low solvent vapour pressure atmosphere.

### FIELD OF THE INVENTION

The present invention relates to methods of the type mentioned in the preamble of the independent claim for preparing samples for analysis thereof.

### PRIOR ART

In microchannel electrophoresis analysis, an analyte, for example a liquid sample containing a mixture of proteins, is introduced into a microchannel in a non-conducting substrate and a voltage is applied across the ends of the channel in order to separate the components of the analyte. The electrical field created by this applied voltage causes the components of the analyte, e.g. the proteins, to be displaced along the length of the channel. There are several modes of utilising this effect to separate the analyte into its components; isoelectric focusing, isotachophoresis, zone electrophoresis, and moving boundary electrophoresis. Once the separation is complete it is often necessary to remove the set of separated analyte components (called the “sample-vector”) from the microchannel so that the components of the sample can be identified, for example by mass spectroscopy. Various mobilisation techniques are available for removing the sample-vector from one end of the microchannel, for example, displacement of the sample-vector by fresh liquid pumped by means of pressure pumping or electrokinetic pumping into one end of the channel or sucking the sample vector out of the microchannel, by electrospray extraction, etc. All these mobilisation techniques suffer from the problem that they inevitably disturb the sample-vector and cause the separated molecules to mix, thereby leading to band-broadening in the subsequent identification stage. In a macroscopic format similar separations are usually performed in a gel that, amongst other properties, reduces unwanted liquid turbulence and flow. After the molecules have been separated over the gel, the gel can be cut into pieces that are analysed individually, or dried as a whole, for subsequent analysis using e.g. MALDI. Among the drawbacks of the macroscopic format is that it is very time-, space- and reagent-consuming. This is especially true when it is being used in true high-throughput proteomics applications.

### SUMMARY OF THE INVENTION

According to the present invention, at least some of the problems with the prior art are solved by means of a method having the features of claim 1. Further improved methods have the features of the dependent claims.

The present invention uses the principle of lyophilisation. Lyophilisation is the process of using sublimation, i.e. evaporation from a solid phase directly into the gas phase, to decrease the content of a solvent, usually water, in a substance.

In a method in accordance with the present invention a sample containing analytes such as proteins and/or peptides is introduced in a liquid form into a micro channel system. The sample can there optionally be subjected to several types of micro-fluidic liquid handling such as pumping, mixing, splitting, metering, temperature treatments, reactions, chromatography, and electrophoresis, etc. which result in the analytes being redistributed in the microchannels, preferably in a controlled manner. The sample liquid is then frozen. The frozen sample is made accessible by removing part of the container, preferably the lid of the microchannel structure. The exposed sample can now optionally be treated by means of introducing other chemicals onto the sample. This can be accomplished e.g. by using spraying or diffusion from the gas phase. Methods that can be used with more precise local selectivity such as stamping or micro-pipetting are also possible. Preferably, these processes are accomplished in such a manner that the sample is only locally thawed or melted in order to reduce movement of the analytes and maintain the preserved distribution. The solvent of the sample liquid is then partially or preferably, substantially completely, sublimated away. This process will leave the analytes on an open surface with virtually the same distribution as when they were in the liquid state following the micro-fluidic handling. If substantially all the solvent has been removed, the analytes will be available in a form suitable for analyses requiring dry and non-encapsulated samples, for example, matrix-assisted laser desorption ionisation mass spectroscopy (MALDI-MS).

As the method is compatible with solutions containing acetonitrile the process is suitable for combination with e.g. reversed phase chromatography (RPC). In a conceivable set up for high-throughput proteomics in accordance with the present invention, the eluent fed onto a miniature RCP-column loaded with a protein sample is mixed with a known set of ampholytes. The proteins pass through an arrangement for on-line digestion and are digested into peptides in the order that they are released from the column. The eluent flow carrying the peptides is fractionated into micro-channels and treated in accordance with the present invention. This would result in a second separation in two-dimension separation process with protein hydrophobicity on the first axis and the constituting peptides separated and concentrated on the second axis on a format suitable for mass spectroscopy identification.

### BRIEF DESCRIPTION OF THE FIGURES

FIG. 1 shows schematically a plan view of a microchannel electrophoresis plate;

FIG. 2 shows a cross-section along line II-II of FIG. 1;

FIGS. 3a)-3g) show schematically stages in a sample-vector transferring method in accordance with a first embodiment of the present invention.

FIG. 4 shows the data collected by an MALDI-TOF mass spectrometer along the length of a microchannel treated according to the present invention. The collected data are counts of molecules in a mass interval corresponding to one of the analytes in the introduced liquid.

### DETAILED DESCRIPTION OF EMBODIMENTS ILLUSTRATING THE INVENTION

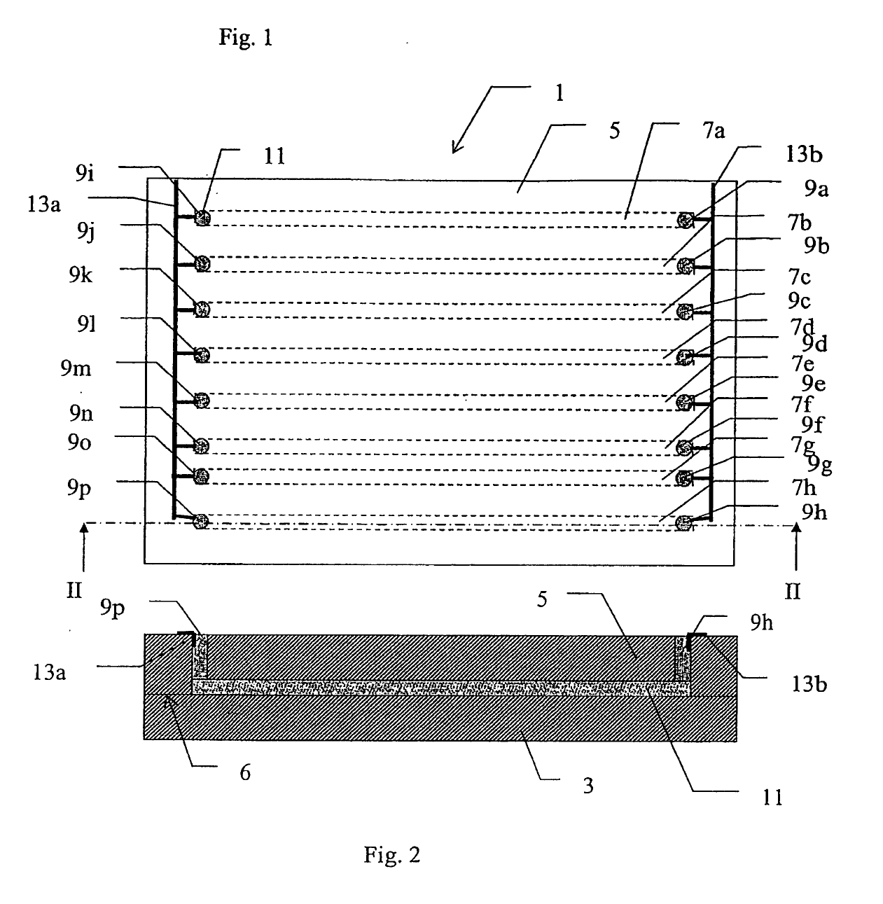

A first embodiment of an arrangement for lyophilisation of microchannels 1 in accordance with the present invention is illustrated schematically in FIG. 1). The arrangement 1 comprises a substrate 3 and a lid 5 reversibly bonded together at a bond interface 6. The substrate 3 and the lid 5 can be made from common materials for micro-liquid handling such as PDMS but the use of other materials and combinations of materials that can be sealed together, spontaneously or by force, is possible. Structures forming at least one microchannel 7a-7h at the interface between the substrate 3 and lid 5 are provided in the substrate 3 and/or the lid 5. Preferably, the substrate 3 and/or the lid 5 have at least one via hole 9a-9p through which the microchannels 7a-7h can be accessed from the exterior.

A first embodiment of a method for preparing a sample for analysis in accordance with the present invention comprises the following steps:

A sample 11 in a liquid form is introduced into one or more of the microchannels 7a-7h.

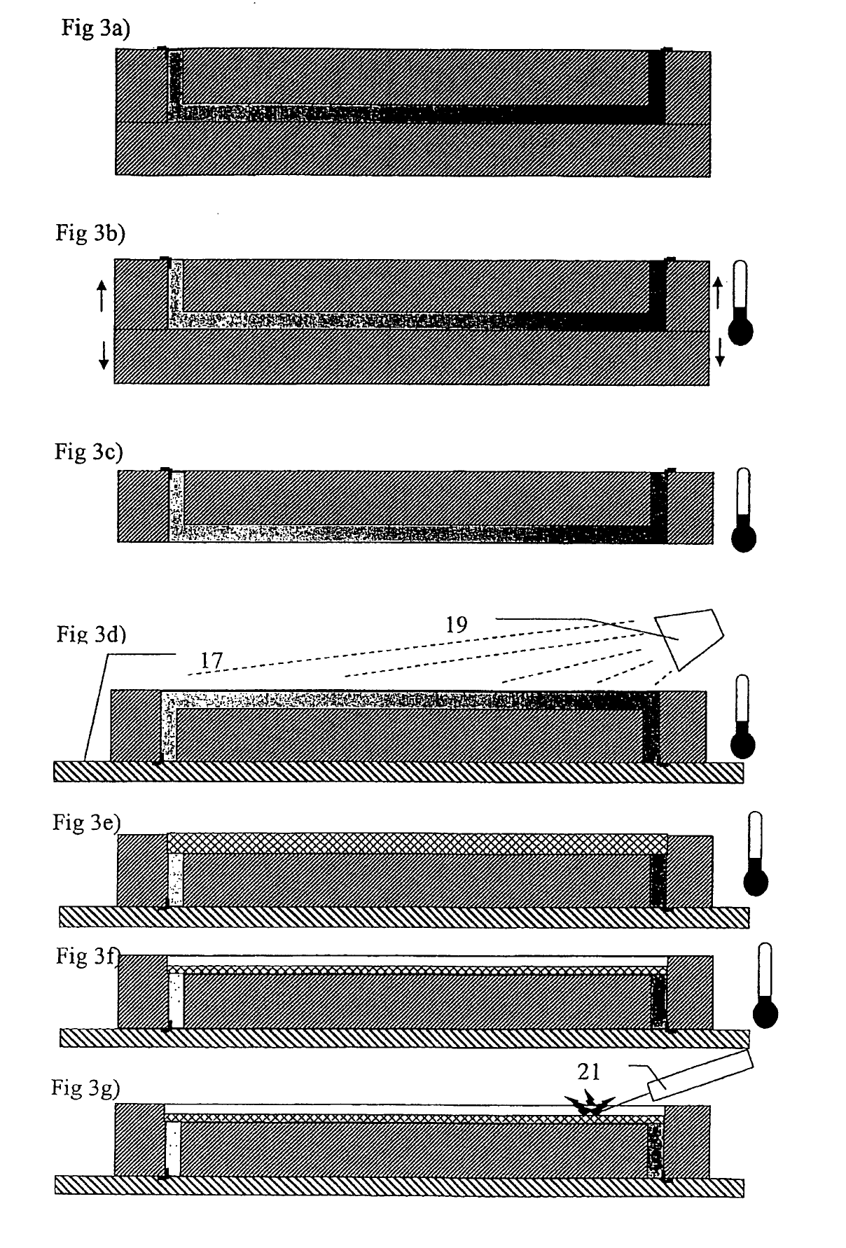

The sample 11 is optionally processed in the microchannels using micro-fluidic liquid handling e.g. the sample molecules are separated by electrophoresis. Depending on the type of processing to be used the microchannels 7a-7h can have different shapes. For example, for zone electrophoresis, a double-T cross is preferred but for isoelectric focusing a simple straight channel will be preferred. More complex structures for more advanced liquid processing are also possible in accordance with the present invention. The use of the channels as sample reservoirs i.e. utilising no micro-fluidic liquid handling subsequent to the introduction is also possible in accordance with the present invention. As an example to illustrate the present invention the use of capillary isoelectric focusing is described. The arrangement 1 may be provided with two electrodes 13a, 13b—one at each end of the microchannels. The electrodes 13a, 13b can be arranged in contact with the introduced sample 11 and can be connected to a power supply (not shown) in order to produce a high electric potential difference (in the order of kilovolts) between them. This potential difference causing amphoteric analytes such as proteins and peptides in the sample 11 to redistribute in the microchannels 7a-7h according to their isoelectric potentials as illustrated schematically in FIG. 3a). As methods for conduct capillary isoelectric focusing are well know to those skilled in the art, no further details on capillary isoelectric focusing will be given.

After any optional liquid handling has been performed the sample 11 is frozen from its liquid state to a frozen state as shown schematically in FIG. 3b) in which a thermometer symbol shows a low temperature. This can be done simply by pouring liquid nitrogen or the like onto the substrate 3 or the lid 5. The temperature of the sample 11 can also be lowered below its freezing temperature using other methods such as the use of a freezer or the like. Other more sophisticated methods, such as pumping of a cool media inside adjacent channels in the substrate 3 or lid 5, are also possible. For the best possible result it is important that the freezing process does not redistribute the sample. This can be ensured when freezing is homogeneously initiated at many places simultaneously causing a fine grained frozen state. Depending on the composition of the sample solution the freezing process may result in a system consisting of more than one phase. For example if the freezing process results in a two-phase system one phase can be in a frozen state and the other phase in a liquid state. If the ratio of the frozen phase is above a certain fraction that depends on the sample constituents the frozen phase can extend continuously throughout the channel structure in a network and thereby virtually inhibit transport of the frozen phase within the channels as well as virtually inhibit redistribution of analytes within the phase. Such a network can also, depending on its tightness, prevent redistribution of the liquid phase as well. When freezing the sample to a frozen state in a preferred method according to the present invention the frozen state consists of at least one frozen phase and that said frozen phase substantially prevents sample transport within the channels.

After freezing the sample 11 the bond interface 6 is at least partially opened so that at least a part of the sample 11 is exposed to the surrounding atmosphere. The properties of the surfaces of the substrate 3 and lid 5 exposed to the sample 11 within the microchannels 7a-7h are preferably chosen so that the frozen sample 11 will preferably remain stuck either on the substrate 3 or on the lid 5. For example, if both the substrate 3 and the lid 5 are made from non-coated PDMS the sample 11 will to a very large extent remain in the microchannels 7a-7h as shown in FIG. 3c).

Once the frozen sample 11 has been exposed the sample 11 is available for additional chemical treatment. In order to illustrate the present invention a possible preparation for MALDI-MS analysis is described. The frozen sample 11 present in the microchannels 7a-7h in the lid 5 can, after inverting of the lid 5, be mixed with MALDI matrix by spray coating with a spray nozzle 19 as shown in FIG. 3d). To minimise movement of the analyte within the sample it is essential that the spraying only thaws and integrates with the sample locally forming a mixture of analyte and matrix as illustrated in FIG. 3e). For example, this can be controlled by placing the lid 5 on a chilled surface 17 or in a cool atmosphere and controlling the amount of deposited spray per area and time in order to minimise melting of the frozen sample.

After having optionally performed any desired post-freezing chemical treatments, the sample solvent vapour pressure of the surrounding atmosphere is lowered sufficiently to obtain a low solvent vapour pressure atmosphere which allows lyphilization, i.e. which allows the solvent/solvents of the sample 11 to sublimate away from the surface on which it is/they are present i.e. the substrate 3 (or the lid 5 as illustrated in FIG. 3f). Solvents of the sample 11 can be for example water, acetonitrile, ethanol or other organic solvents. A low solvent vapour pressure atmosphere can be achieved by means of applying vacuum or a nitrogen gas to a container containing the sample 11. One way to lower the solvent vapour pressure is to use a so-called freeze-dryer where the solvent vapour is condensed and frozen on a chilled plate in a typically low-pressure atmosphere. To speed up the process freeze-dryers are usually equipped with a substrate heater, however in the present invention the heater is preferably not used. If a heater is used then it is preferably controlled such that the solvent sublimates but the sample does not melt—this is in order to prevent movement of the analytes.

After the sample has been lyophilized to a state of sufficiently low content of solvent, i.e. the solvent content of the sample 11 is so low that the sample will not redistribute in a manner detrimental to the analysis because of liquid transport upon thawing, the temperature may preferably raised. Preferably the temperature is raised in a dry environment so that any water or solvent vapour in the atmosphere does not condense to an injurious extent onto the sample 11.

In order to achieve the least movement of analytes from the positions that they reached during the liquid handling, it is desirable that the sample 11 does not melt completely during any stage of the lyophilization process.

After the sample has been lyophilized and the temperature has been raised, the sample 11 is available for subsequent analysis using e.g. MALDI-TOP (using a laser 21 as illustrated in FIG. 3g), fluorescence scanning, NMR scanning, etc.

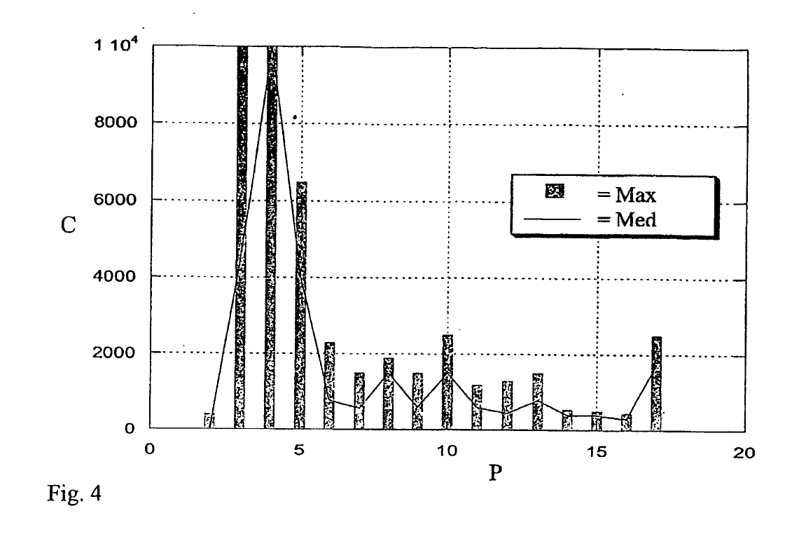

In an experiment conduced in accordance with the present invention, a sample containing 40% Acetonitrile, 10 μM Angiotensine, 10 μM Bradykinine, 4 mM Arginine and 4 mM Glutamic acid in water was introduced in several parallel 70 mm long 350 μm wide and about 50 μm deep microchannels formed in a substrate and covered by a removable lid with vias leading to the microchannels. The sample was treated using electrophoresis in a mode similar to a so-called isoelectric group-focusing mode using 0.1M H₃PO₄ as anolyte and 0.02M NaOH as catholyte and with a voltage applied over the length of the microchannels that was raised from 0V to 1000V over a period of 88 seconds. Pouring liquid nitrogen over the lid then froze the sample. The lid was removed which also removed the anolyte and catholyte present in the vias, leaving the major part of the sample in the microchannels formed in the substrate. The substrate with the frozen sample was placed in a container through which cool gas exiting from a frozen nitogen reservoir was forced using a small fan. The substrate was sprayed with saturated α-cyano-4-hydroxycinnamic acid in a solution of 49.9% water, 50% Acetonitrile, and 0.1% Trifluoroacetic acid using a spray needle moved by a robot arm. The robot arm was moved at a speed of 32 mm/s over the substrate and 16 μl/min of the spray solution was deposited in two passes on the substrate leaving an approx. 2 mm wide line of MALDI matrix. The substrate was then left in a box placed in a freezer at approx. −20° C. and the box was flushed with nitrogen gas having the same temperature as freezer. The box was left for several hours and the box was then brought to room temperature before the nitrogen flush was terminated. The substrate was placed in an MALDI-TOF and spectra were taken along one of the microchannels. Counts (C) in a selected mass interval corresponding to Bradykinine are presented in FIG. 4. Note that the detector has been saturated at positions (P) 3 and 4. The bar on the graph show the maximum counts (Max) and the lines show the median (Med) counts at each position P on the substrate.

In an alternative embodiment of the present invention, the frozen sample is removed from the substrate in which the sample was separated and transferred to a second substrate, for example a specially treated MALDI slide. This can be achieved by removing the lid from the substrate, placing the newly exposed surface of the substrate in contact with a surface on the second substrate, warming the substrate on the surface facing away from the second substrate so as to loosen the frozen sample from the channels in the substrate and, optionally, cooling the second substrate so that the sample freezes to its surface. Once the sample has been loosened from the microchannels the substrate can be lifted away from the second substrate, leaving the sample on the second substrate.

The above mentioned embodiments are intended to illustrate the present invention and are not intended to limit the scope of protection claimed by the following claims.

1. A method for reducing solvent content of a sample in a microchannel or other microfluidic structure comprising: 1) freezing said sample and 2) exposing the frozen sample to a low solvent vapour pressure atmosphere.

2. The method of claim 1, wherein step 2) comprises 2a) removing a lid from said microchannel or microfluidic structure to expose said frozen sample and 2b) providing a low solvent vapour pressure atmosphere over the exposed frozen sample.

3. The method of claim 2, further comprising depositing or incorporating additional chemical components onto the frozen sample prior to or simultaneously with exposing said sample to said low solvent vapour pressure atmosphere.

4. The method of claim 1, wherein said sample is electrophoretically separated prior to step 1).
